(19) United States
(12) Patent Application Publication
(10) Pub. No.: US 2018/0014739 A1
(43) Pub. Date: Jan. 18, 2018

(54) UNOBTRUSIVE EMOTION RECOGNITION SYSTEM

(71) Applicant: Sentio Solutions, Inc., San Francisco, CA (US)

(72) Inventors: Georgios Eleftheriou, Santa Clara, CA (US); Panagiotis Fatouros, Athens (GR); Charalampos Tsirmpas, Athens (GR)

(21) Appl. No.: 15/648,730

(22) Filed: Jul. 13, 2017

Related U.S. Application Data
(60) Provisional application No. 62/361,808, filed on Jul. 13, 2016.

(51) Int. Cl. A61B 5/0476; G06K 9/00; H05K 7/02; G06F 17/30; A61B 5/16; G06F 19/00; A61B 5/00

(52) U.S. Cl. CPC A61B 5/0476; A61B 5/165; G06K 9/00335; G06F 19/3406; H05K 7/02; G06F 17/30017; A61B 5/00; G06F 19/00

(57) ABSTRACT

One variation of a system for unobtrusively recognizing emotions of users includes: a galvanic skin response sensor; a heart rate sensor; a skin temperature sensor; a motion sensor; and a controller configured to sample a sequence of biosignal values of a user from the galvanic skin response sensor, the heart rate sensor, the skin temperature sensor, and the motion sensor, to implement statistical tests, self-organising maps, and clustering techniques to identify of significant changes in characteristics of the sequence of biosignal values, and to predict an emotional status of the user based on the significant changes in characteristics of the sequence of biosignal values.

### CROSS-REFERENCE TO RELATED APPLICATIONS

This application claims the benefit of U.S. Provisional Application No. 62/361,808, filed on 13 Jul. 2016, which is incorporated in its entirety by this reference.

### TECHNICAL FIELD

This invention relates generally to the field of emotion sensing and more specifically to a new and useful unobtrusive emotion recognition system in the field of emotion sensing.

### BACKGROUND

In today's intense and stressful society, all of us lose track of our emotions. We spend long periods of time experiencing negative feelings without even realizing it. Those negative feelings and stress affect physiological and psychological health. It is noteworthy that 25% of the population is suffering from depression and anxiety according to World Health Organization. Recognizing your stress triggers or what affects your mood is the first step in conquering stress and improving your well-being. At the same time, understanding and reinforcing the habits that make you feel good can increase your happiness. This requires the hardware and the software able to monitor unobtrusively physiological signals such as Electrodermal Activity (EDA), Blood Volume Pulse, etc. for a long period of time and recognize different emotional states.

### BRIEF DESCRIPTION OF THE FIGURES

FIG. 1 is a flow chart representation of a system;

FIG. 2 is a schematic representation of one variation of the system;

FIG. 3 is a schematic representation of one variation of the system;

FIG. 4 is a schematic representation of one variation of the system.

### DESCRIPTION OF THE EMBODIMENTS

The following description of embodiments of the invention is not intended to limit the invention to these embodiments but rather to enable a person skilled in the art to make and use this invention. Variations, configurations, implementations, example implementations, and examples described herein are optional and are not exclusive to the variations, configurations, implementations, example implementations, and examples they describe. The invention described herein can include any and all permutations of these variations, configurations, implementations, example implementations, and examples.

A system 100 for unobtrusively recognizing emotions of users includes: a galvanic skin response sensor; a heart rate sensor; a skin temperature sensor; a motion sensor; and a controller configured to sample a sequence of biosignal values of a user from the galvanic skin response sensor, the heart rate sensor, the skin temperature sensor, and the motion sensor, to implement statistical tests, self-organising maps, and clustering techniques to identify of significant changes in characteristics of the sequence of biosignal values, and to predict an emotional status of the user based on the significant changes in characteristics of the sequence of biosignal values.

Generally, a problem with real life unobtrusive emotion recognition is that the acquisition device placement and ergonomy and electrode material and placement can heavily impact measurements and produce less meaningful biosignal readings. Consequently, they should be designed taking into account both the quality of the acquired physiological signals and the user comfort.

Another problem with the Electrodermal Activity (EDA) signal in non-controlled environments is that it can contain little-to-no physiological information when a number of factors are ignored. Motion of the user can create unwanted effects, such as displacement of the electrodes or squeezing of the skin against the electrodes. Further, changes in ambient temperature and/or humidity may alter baseline EDA readings. Sensors must compensate for all of these factors.

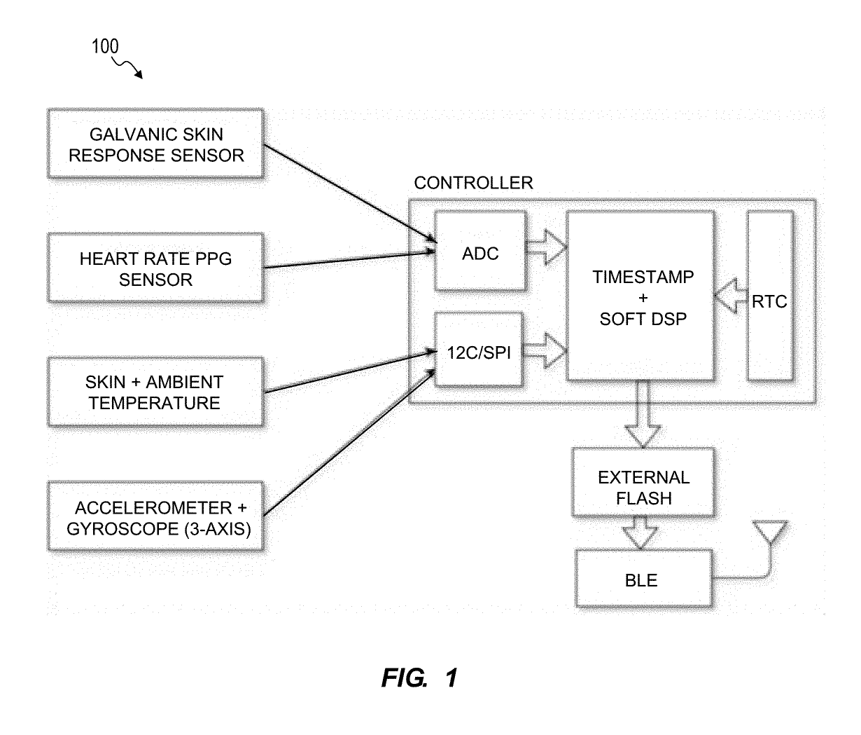

In an exemplary implementation of this invention, the Main Board is based around an ARM Cortex-M microcontroller, with a wide set of integrated features, including Analog-to-Digital Converter and a Real-Time Clock (FIG. 1). An IMU sensor communicates with the MCU via two/three-wire protocol, such as the ADXL345 from Analog Devices. A PPG sensor, capable of extracting the user's heart rate may feature a number of LEDs and a photodetector. A thermopile temperature sensor provides contactless skin temperature readings and transmits them to the MCU via I2C/SPI protocol. Proper Analog Front End, including off-the-self operational amplifiers (e.g. Analog Devices AD8606), are used for the pre-processing of the analog bio-signals before their digital conversion. The system is battery powered and a battery charging IC is featured in the main board (e.g. Maxim 8814). The battery itself is a Lithium Ion/Polymer battery with a medium capacity and integrated protection circuitry.

The wearable device is acquiring a number of bio-signals at a given frequency and logs them to the MCU's onboard flash. When the integrated flash is almost full, the data are logged to an external non-volatile chip (Flash, FRAM or other technology). Once in a while, the BLE module syncs with the mobile application and wirelessly transmits the logged data, for further processing and visualization.

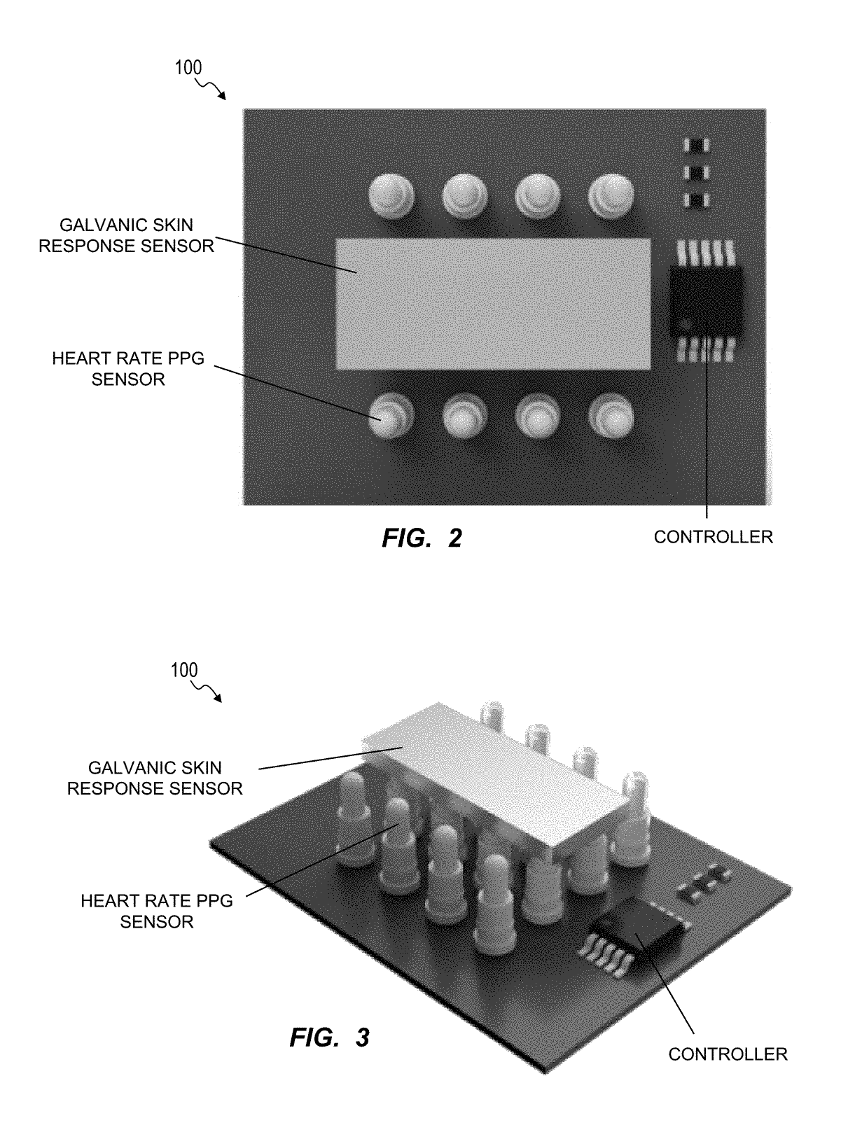

In an exemplary implementation of this invention, in order to reduce the effect of various artifacts (motion, pressure, etc.) in EDA signal, a module as seen in FIG. 2 and FIG. 3 is realized. In this exemplary module, low force, spring-loaded electrodes are used, capable of following the user's skin movement. In this way, user's motion does not cause electrode squeezing against the skin, thus not disturbing stratum coneum's properties. The array of electrodes receives additional information regarding the percentage of contact surface and the respective pressure applied to it. These electrodes may switch on and off in high sample rate according to the firmware, thus creating an Electrodermal mapping of a given surface of the user's. This enables the device to compensate more accurately for motion and pressure artifacts in biosignals, than relying only in IMU data. This includes high frequency peaks in both GSR and BVP signals, level changes in GSR, etc.

A problem with real life unobtrusive emotion recognition through physiological measurements (Galvanic Skin Response, Blood volume pulse, Skin Temperature etc.) is that the autonomic nervous system does not respond to emotional stimulus consistently across different individuals, rather is a deeply personalized process and is characterized by both time and subject context drift.

Additionally, a problem with real life unobtrusive emotion recognition, even regarding a specific individual, is that the beginning, the end and the duration of an emotion experiencing period cannot be explicitly defined, rendering, thus, the identification of an emotional event and the emotion recognition process rather ambiguous.

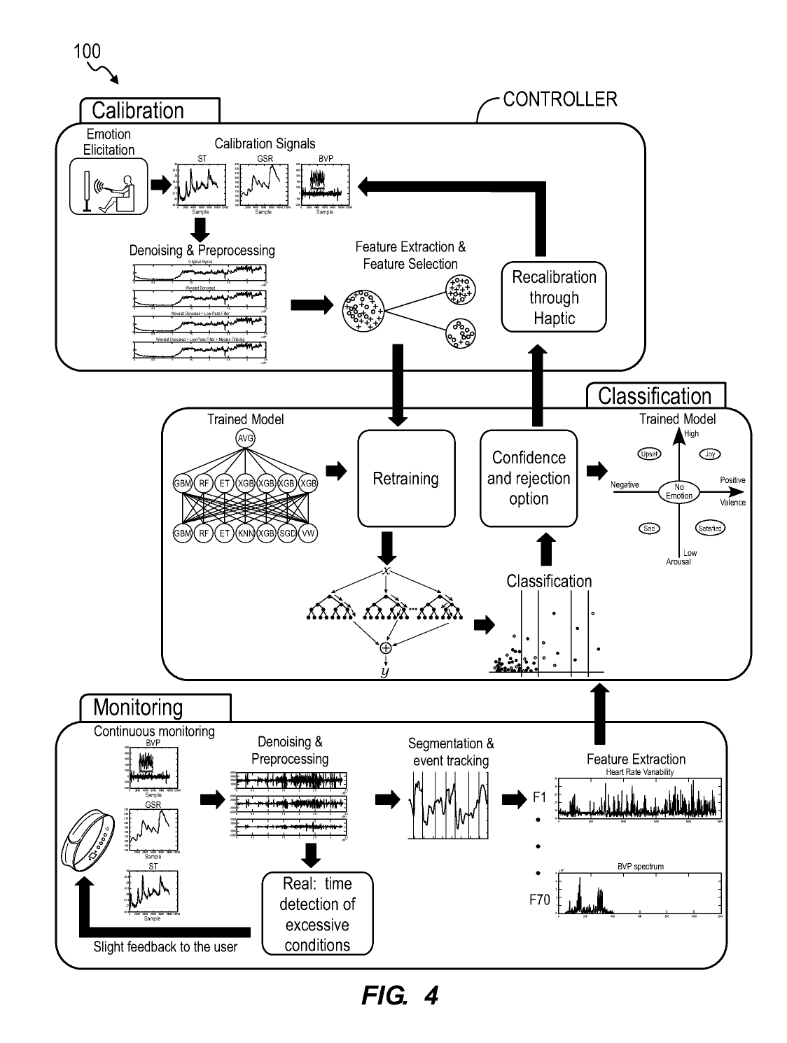

FIG. 4 illustrates a functional diagram of the real-life emotion recognition process, which consists of the calibration, monitoring and classification modules. During the calibration process, the acquisition device collects the physiological signals and calibrates a model to the specific user. In the monitoring process, which corresponds to any real-life application, the acquisition device continuously collects the required signals and comes up with the emotion experiencing intervals. Finally, in the classification module, the individually calibrated model is fused with a unified one and provide the final decision, regarding the emotional state of the user during the previously identified intervals.

In an exemplary implementation of this invention, during the emotion elicitation process, the main objective is to stimulate a sequence of different emotions to the subject and identify their impact on the specific user's biosignals. For this reason, a consistent and reliable protocol is utilised, which guarantees both the isolation of the user during the stimulation process and and maximizes his/her engagement.

In an exemplary implementation of this invention, the acquisition device employed, collects the physiological signals of the user during the emotion elicitation process. The device integrates several biosensors (GSR, BVP, ST etc.) and a motion sensor (inertial measurement unit (IMU)).

In an exemplary implementation of this invention, the acquisition device uses various pre-processing and denoising techniques, for example window smoothing appliance, filtering, motion artifact removal, GSR decomposition and normalisation, to compensate for the complexities introduced by the real-life continuous acquisition. In the filtering stage, a combination of filters, such as lowpass, bandpass, window smoothing and moving average ones is used in order to compensate for any artifacts generated. Additionally, adaptive motion artifact reduction and wavelet filtering schemes are applied to recover the underlying physiologic information from the measured signal, using data from the accelerometer as a motion reference.

In an exemplary implementation of this invention, during the feature extraction process, for an uncontrolled real-life implementation, apart from the usual linear biosignal features, several nonlinear features at the time and frequency domain are extracted from each acquired biosignal and are fused, leading to a total of more than 170 features.

In an exemplary implementation of this invention, advanced dynamic feature selection techniques combined with dynamic voting and ensemble algorithms are incorporated in order to evaluate the the importance of each feature and derive the most suitable feature subset for each individual.

In an exemplary implementation of this invention, during the monitoring process, every time an unusual/excessive event is observed, always taking into account what is considered usual for the specific user, the user is notified by the device in a discrete way (ie. a nudge), which aims to enhance his awareness and lower his arousal levels.

In an exemplary implementation of this invention, since the beginning, the end and the duration of each emotional event is random and not known beforehand, a combination of statistical tests, self-organising maps and clustering techniques are employed for the identification of significant changes in the biosignals characteristics. Firstly, a number of statistical tests are applied on the biosignals in order to identify changes that may correspond to emotional events and then a combination of different clustering techniques is used to bring together segments with similar characteristics.

In an exemplary implementation of this invention, during the retraining process a unified trained model, is retrained and adapted to each individual user, based on the emotional data acquired during the calibration process. The combination of the data obtained from each individual user and the knowledge extracted from a big pool of different users, significantly improves the performance of the algorithms and the sophistication of the final decision. The personalized trained model will constitute the personal inference engine.

In an exemplary implementation of this invention, the inference engine provides the final outcome regarding the emotional status of the user during the monitoring phase based on the continuously acquired real-time biosignal. Since, the emotional status of the user cannot be crisply defined, the techniques employed at this stage are based on fuzzy principles and combination of different classification techniques (ie. fuzzy random forests, fuzzy svm, etc.).

In an exemplary implementation of this invention, since real-life applications require increased attention, the classification membership values should be evaluated, before the final decision is communicated. Results that are either ambiguity rejected or membership rejected, are not deemed accurate enough to be communicated to the user. Consequently, only results that exhibit high membership values or are characterized by high confidence levels, will be output.

In an exemplary implementation of this invention, in case of decisions being either membership or ambiguity rejected, the user will be notified and urged to provide haptic feedback through the acquisition device regarding his emotional status during a specific time interval and the personalized model will be updated and recalibrated real time, based on the feedback.

The systems and methods described herein can be embodied and/or implemented at least in part as a machine configured to receive a computer-readable medium storing computer-readable instructions. The instructions can be executed by computer-executable components integrated with the application, applet, host, server, network, website, communication service, communication interface, hardware/firmware/software elements of a user computer or mobile device, wristband, smartphone, or any suitable combination thereof. Other systems and methods of the embodiment can be embodied and/or implemented at least in part as a machine configured to receive a computer-readable medium storing computer-readable instructions. The instructions can be executed by computer-executable components integrated by computer-executable components integrated with apparatuses and networks of the type described above. The computer-readable medium can be stored on any suitable computer readable media such as RAMs, ROMs, flash memory, EEPROMs, optical devices (CD or DVD), hard drives, floppy drives, or any suitable device. The computer-executable component can be a processor but any suitable dedicated hardware device can (alternatively or additionally) execute the instructions.

As a person skilled in the art will recognize from the previous detailed description and from the figures and claims, modifications and changes can be made to the embodiments of the invention without departing from the scope of this invention as defined in the following claims.

I claim:

1. A system for unobtrusively recognizing emotions of users comprising:
a galvanic skin response sensor;
a heart rate sensor;
a skin temperature sensor;
a motion sensor; and
a controller configured to:
sample a sequence of biosignal values of a user from the galvanic skin response sensor, the heart rate sensor, the skin temperature sensor, and the motion sensor;
implement statistical tests, self-organising maps, and clustering techniques to identify of significant changes in characteristics of the sequence of biosignal values; and
predict an emotional status of the user based on the significant changes in characteristics of the sequence of biosignal values.
